(19) United States
(12) Patent Application Publication
(10) Pub. No.: US 2026/0102563 A1
(43) Pub. Date: Apr. 16, 2026

(54) SYSTEMS AND METHODS FOR INSULIN INFUSION

(71) Applicant: ENDOCRINE TECHNOLOGIES, LLC, Scottsdale, AZ (US)

(72) Inventors: Atul Lalani, Scottsdale, AZ (US); Benjamin Lalani, Scottsdale, AZ (US)

(21) Appl. No.: 19/334,370

(22) Filed: Sep. 19, 2025

Related U.S. Application Data
(60) Provisional application No. 63/706,963, filed on Oct. 14, 2024.

(51) Int. Cl. A61M 5/168; A61M 5/172; G16H 10/60

(52) U.S. Cl. CPC A61M 5/16877; A61M 5/1723; G16H 10/60; A61M 2202/07; A61M 2205/3584; A61M 2205/502; A61M 2205/702; A61M 2230/201

(57) ABSTRACT

A method of administering insulin to a patient to achieve and maintain euglycemia is disclosed. The method includes administering insulin to the patient at a drip rate that is based on a current blood glucose level of the patient and a weight of the patient. Insulin is administered during an initial phase, a transition phase (during which blood glucose level decreases slower than during the initial phase), and a maintenance phase. During the maintenance phase, the method includes adjusting the drip rate periodically based on a current drip rate and a percentage change in blood glucose level during a period of time. In addition, the maintenance phase considers a lookback period in which the drip rate is adjusted based on trends during the lookback period.

### RELATED APPLICATIONS

This application claims the benefit of U.S. Provisional Ser. No. 63/706,963, filed Oct. 14, 2024 to Lalani et al., titled “System and Method for Insulin Infusion,” the entirety of the disclosure of which is hereby incorporated by this reference. This application also incorporates by reference the subject matter disclosed in U.S. Design Ser. No. 30/024,085 , filed Sep. 19, 2025, and titled “DISPLAY SCREEN OR PORTION THEREOF WITH GRAPHICAL USER INTERFACE.”

### TECHNICAL FIELD

This document relates generally to insulin infusion, and more specifically to systems and methods for insulin infusion in a critical care setting to reduce the occurrence of hyperglycemia and hypoglycemia and maintain blood sugars within a safe range.

### BACKGROUND

Over the past decades, it has been proven beyond doubt that control of hyperglycemia in critically ill patients is associated with improved outcomes in all patients exhibiting hyperglycemia, whether those patients are diabetic or not. For critically ill patients, there are several factors that result in hyperglycemia, including stress of illness, catecholamines release, and use of medication (such as corticosteroids, vasopressors, etc.), among other factors.

To combat hyperglycemia, the use of insulin infusions with frequent monitoring of blood sugars and adjustment of the dosage accordingly offers a distinct advantage over other forms of therapies and routes of administration due to its short half-life in the IV infusions. The short half-life allows for alterations in the insulin dosage to match the glucose variances that are common during critical illness. At the same time, while it is important to control hyperglycemia, overzealous use of insulin can potentially be as harmful if it leads to hypoglycemia. Thus, keeping the sugar levels in a tight and safe range is essential for better outcomes while treating the cause of the critical illness.

### SUMMARY

According to some embodiments, the present disclosure relates to a method of administering insulin to a patient to achieve and maintain euglycemia. In some embodiments, the method includes administering insulin to the patient at a drip rate that is based on a current blood glucose level of the patient and a weight of the patient. In some embodiments, during an initial phase, the method includes adjusting the drip rate periodically until a blood glucose level of the patient decreases to a predetermined blood glucose level above euglycemia. In some embodiments, adjusting the drip rate during the initial phase includes repeatedly determining a target blood glucose level for time Tₙ based on the current drip rate, observing the blood glucose level at time Tₙ, comparing the observed blood glucose level with the predetermined blood glucose level, comparing the observed blood glucose level with the target blood glucose level, and calculating an updated drip rate based on the comparison of the observed blood glucose level with the target blood glucose level. In some embodiments, during a transition phase, the method includes adjusting the drip rate such that the blood glucose level of the patient decreases slower during the transition phase than the initial phase. In some embodiments, the method includes after the blood glucose level reaches euglycemia during the transition phase, entering a maintenance phase. In some embodiments, during the maintenance phase, the drip rate is adjusted periodically based on a current drip rate and a percentage change in blood glucose level during a period of time. In some embodiments, during the maintenance phase, the method includes evaluating all blood glucose levels of the patient measured that occurred during a lookback period, determining whether each successive blood glucose level measurement moved in a same direction, determining a cumulative magnitude of change in blood glucose level during the lookback period, and determining a current blood glucose level. In some embodiments, the method includes increasing the drip rate if each successive blood glucose level measurement during the lookback period increased, the current blood glucose level is above a predetermined level, and the cumulative magnitude of change is above a predetermined magnitude. In some embodiments, the method includes decreasing the drip rate during the lookback period if each successive blood glucose level measurement during the lookback period decreased, the current blood glucose level is below a predetermined level, and the cumulative magnitude of change is above a predetermined magnitude.

In some embodiments, the lookback period is a four-period period. In some embodiments, the predetermined blood glucose level is 200 mg/dL. In some embodiments, an amount of time in the initial phase is predetermined. In some embodiments, the initial phase lasts four hours. In some embodiments, an amount that the blood glucose level of the patient decreases during the initial phase is limited based on an osmolality of the patient. In some embodiments, a basal insulin dosage for subcutaneous delivery is calculated at an end of the maintenance phase.

In some embodiments, a system for administering insulin to a patient to achieve and maintain euglycemia includes a server communicatively coupled to a user device, a blood glucose meter, an electronic health records database, and an insulin IV pump. The system may also include a processor communicatively coupled to the server. The processor is configured to receive user inputs from the user device, receive a blood glucose reading from the blood glucose meter, receive electronic health records from the electronic health records database, and send a drip rate instruction to the insulin IV pump. In some embodiments, the drip rate instruction to the insulin IV pump includes an initial drip rate instruction, an initial phase instruction, a transition phase instruction, and a maintenance phase instruction. The initial drip rate instruction may be based on a current blood glucose reading received from the blood glucose meter and a patient weight received from the user device. In some embodiments, the initial phase instruction includes periodically adjusting the drip rate by determining a target blood glucose level for time Tn based on a current drip rate, receiving an observed blood glucose level at time Tn, comparing the observed blood glucose level with a predetermined blood glucose level above euglycemia, comparing the observed blood glucose level with the target blood glucose level, and calculating an updated drip rate based on the comparison of the observed blood glucose level with the target blood glucose level.

In some embodiments, the transition phase instruction includes adjusting the drip rate so that the blood glucose level of the patient decreases at a slower rate during the transition phase than the initial phase. In some embodiments, the maintenance phase instruction involves adjusting the drip rate periodically based on the current drip rate and a percentage change in blood glucose level during a period of time. During the maintenance phase, the processor evaluates all blood glucose levels of the patient measured during a lookback period, determines whether each successive blood glucose level measurement moved in the same direction, determines a cumulative magnitude of change in blood glucose level during the lookback period, and determines the current blood glucose level.

In some embodiments, the processor increases the drip rate if each successive blood glucose level measurement during the lookback period increased, the current blood glucose level is above a predetermined level, and the cumulative magnitude of change is above a predetermined magnitude. Conversely, in some embodiments, the processor decreases the drip rate if each successive blood glucose level measurement during the lookback period decreased, the current blood glucose level is below a predetermined level, and the cumulative magnitude of change is above a predetermined magnitude.

In some embodiments, a system for administering insulin to a patient to achieve and maintain euglycemia includes a server communicatively coupled to a user device, a blood glucose meter, an electronic health records database, and an insulin IV pump. The system may also include a processor communicatively coupled to the server. The processor is configured to receive user inputs from the user device, receive a blood glucose reading from the blood glucose meter, receive electronic health records from the electronic health records database, and send a drip rate instruction to the insulin IV pump. In some embodiments, the drip rate instruction includes an initial drip rate instruction, an initial phase instruction, a transition phase instruction, and a maintenance phase instruction. The initial drip rate instruction may be based on a current blood glucose reading and a patient weight. The initial phase instruction may include periodically adjusting the drip rate by determining a target blood glucose level for time Tn, receiving an observed blood glucose level at time Tn, comparing the observed blood glucose level with a predetermined blood glucose level above euglycemia, comparing the observed blood glucose level with the target blood glucose level, and calculating an updated drip rate based on the comparison. The transition phase instruction may include adjusting the drip rate so that the blood glucose level decreases at a slower rate than during the initial phase. The maintenance phase instruction may involve adjusting the drip rate periodically based on the current drip rate and a percentage change in blood glucose level during a period of time. During the maintenance phase, the processor may evaluate all blood glucose levels measured during a lookback period, determine whether each successive measurement moved in the same direction, determine a cumulative magnitude of change, and determine the current blood glucose level. The processor may increase the drip rate if all measurements increased, the current blood glucose level is above a predetermined level, and the cumulative magnitude of change is above a predetermined magnitude. Conversely, the processor may decrease the drip rate if all measurements decreased, the current blood glucose level is below a predetermined level, and the cumulative magnitude of change is above a predetermined magnitude.

In some embodiments, the processor is further configured to query the electronic health records database to determine if the patient has received a basal insulin formulation within the previous 24 hours. In some embodiments, if the previous dose was administered within the last 24 hours, the processor instructs the IV pump to administer an initial basal dose at a set time. If the previous dose was not administered within the last 24 hours, the processor instructs the IV pump to administer a prorated basal dose and subsequently administer the initial basal dose at the set time. In some embodiments, the processor is configured to receive a patient osmolality. In some embodiments, the processor is configured to adjust the dosage based on the received patient osmolality. In some embodiments, the lookback period comprises a four-period period.

In some embodiments, the processor is further configured to generate a graphical user interface having a first section displaying patient-specific data and a second section displaying a chart of the drip rate over time.

In some embodiments, a method of administering insulin to a patient to achieve and maintain euglycemia includes receiving patient-specific data, determining an initial carbohydrate ratio and an initial insulin sensitivity factor based on the patient-specific data, calculating a total daily insulin dose based on the patient-specific data, dividing the total daily insulin dose into a bolus component and a basal component, the basal component comprising an initial basal dose, periodically updating the carbohydrate ratio and the insulin sensitivity factor based on an observed insulin response, carbohydrate intake, and a lookback period, and continuously adjusting the bolus component in response to changes in carbohydrate intake and the observed insulin sensitivity.

In some embodiments, the lookback period comprises a four-period period. In some embodiments, the patient-specific data comprises weight, body mass index, current blood glucose level, comorbidities, and information regarding concurrent medications.

In some embodiments, a method of administering insulin to a patient to achieve and maintain euglycemia involves several steps, including, first, a processor receives patient-specific data, including weight, body mass index, current blood glucose level, comorbidities, and information regarding concurrent medications. Based on this data, the processor may calculate a total daily insulin dose. In some embodiments, the processor may divide the insulin dose into a basal component and a bolus component. The basal component may be designed to maintain baseline glucose levels. The bolus component may account for carbohydrate intake and glucose correction.

In some embodiments, the processor determines an initial carbohydrate ratio and insulin sensitivity factor for the patient based on the patient-specific data. In some embodiments, the processor is configured to periodically update the carbohydrate ratio and insulin sensitivity factor based on observed insulin response and variability in carbohydrate intake. In some embodiments, the bolus insulin dose is adjusted in response to changes in carbohydrate intake, pre-meal blood glucose measurements, and observed insulin sensitivity. In some embodiments, the patient's blood glucose levels are continuously monitored to maintain glucose within a predetermined target range while minimizing the risk of hypoglycemia. In some embodiments, updates to dosing parameters are made in real-time in response to observed changes in insulin sensitivity and carbohydrate ratio.

The foregoing and other aspects, features, and advantages will be apparent from the DESCRIPTION and DRAWINGS, and from the CLAIMS if any are included.

### BRIEF DESCRIPTION OF THE DRAWINGS

Implementations will hereinafter be described in conjunction with the appended and/or included DRAWINGS.

FIG. 1 shows a schematic illustration of a method of insulin infusion according to some embodiments.

FIG. 2 shows a schematic illustration of an initial phase of a method of insulin infusion according to some embodiments.

FIG. 3 shows a schematic illustration of a transition phase of a method of insulin infusion according to some embodiments.

FIG. 4 shows a schematic illustration of a maintenance phase of a method of insulin infusion according to some embodiments.

FIG. 5 shows a schematic illustration of a system of insulin infusion according to some embodiments.

FIG. 6 shows a schematic illustration of a method of administering a personalized insulin infusion according to some embodiments.

FIG. 7 shows a control table for use with a system or method of administering a personalized insulin infusion according to some embodiments.

FIG. 8 shows a schematic illustration of the preliminary phases of administering a personalized insulin infusion according to some embodiments.

FIG. 9 shows a schematic illustration of administering a personalized insulin infusion according to some embodiments.

FIG. 10 shows a schematic illustration of a system of insulin infusion according to some embodiments.

FIG. 11 shows a schematic illustration of a system of insulin infusion according to some embodiments.

### DETAILED DESCRIPTION

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that embodiments of the technology disclosed herein may be practiced without these specific details. It should be noted that there are many different and alternative configurations, devices and technologies to which the disclosed technologies may be applied. The full scope of the technology disclosed herein is not limited to the examples that are described below.

The singular forms “a,” “an,” and “the” include plural referents unless the context clearly dictates otherwise. Thus, for example, reference to “a step” includes reference to one or more of such steps.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented but have been omitted for purposes of brevity.

The term “plurality,” as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

Throughout the description and claims of this specification, the words “comprise” and “contain” and variations of the words, for example “comprising” and “comprises,” mean “including but not limited to”and are not intended to (and do not) exclude other components.

As required, detailed embodiments of the present disclosure are included herein. It is to be understood that the disclosed embodiments are merely exemplary of the invention that may be embodied in various forms. Therefore, specific structural and functional details disclosed herein are not to be interpreted as limits, but merely as a basis for teaching one skilled in the art to employ the present invention. The specific examples below will enable the disclosure to be better understood. However, they are given merely by way of guidance and do not imply any limitation.

The present disclosure may be understood more readily by reference to the following detailed description taken in connection with the accompanying figures and examples, which form a part of this disclosure. It is to be understood that this disclosure is not limited to the specific materials, devices, methods, applications, conditions, or parameters described and/or shown herein, and that the terminology used herein is for the purpose of describing particular embodiments by way of example only and is not intended to be limiting of the claimed inventions.

More specifically, this disclosure, its aspects and embodiments, are not limited to the specific material types, components, methods, or other examples disclosed herein. Many additional material types, components, methods, and procedures known in the art are contemplated for use with particular implementations from this disclosure. Accordingly, for example, although particular implementations are disclosed, such implementations and implementing components may comprise any components, models, types, materials, versions, quantities, and/or the like as is known in the art for such systems and implementing components, consistent with the intended operation.

Over the past decades it has been proven beyond doubt that control of hyperglycemia in critically ill patients is associated with improved outcomes in all patients exhibiting hyperglycemia, whether those patients are diabetic or not. For critically ill patients, there are several factors that result in hyperglycemia, including stress of illness, catecholamines release, and use of medication (such as corticosteroids, vasopressors, etc.), among other factors.

To combat hyperglycemia, the use of insulin infusions with frequent monitoring of blood sugars and adjustment of the dosage accordingly offers a distinct advantage over other forms of therapies and routes of administration due to its short half-life in the IV infusions. The short half-life allows for alterations in the insulin dosage to match the glucose variances that are common during critical illness. At the same time, while it is important to control hyperglycemia, overzealous use of insulin can potentially be as harmful if it leads to hypoglycemia. Thus, keeping the sugar levels in a tight and safe range is essential for better outcomes while treating the cause of the critical illness. In some embodiments, for example, a target range of blood sugars between 70-180 mg/dL is aimed for. In some embodiments, an additional narrower target range of 100-140 mg/dL is aimed for, which has been associated with even better outcomes as long as there is no increase in the incidence of significant hypoglycemia. Other target ranges may be used in some embodiments.

The present disclosure provides a novel, dynamic method of insulin infusion to achieve euglycemia quicker and to maintain euglycemia once achieved. The disclosed method of insulin infusion may reduce the occurrence of hyperglycemia and hypoglycemia and maintain blood sugars within a safe range. The disclosed method may be used for patients in a critical care setting. The method, in some embodiments, uses artificial intelligence technology to be adaptive to each patient and truly follow the patient's differing requirements and adjust accordingly. The ability to achieve a quicker time to euglycemia and maintain euglycemia once achieved can improve the mean glucose thus reducing glucose variances while minimizing hypoglycemic as well as hyperglycemic events. As a result, the method offers an improved chance of better outcomes to patients that exhibit hyperglycemia requiring intravenous insulin during critical illness.

In some embodiments, the method of insulin infusion comprises one or more phases. For example, the method may include three phases or four phases. In some embodiments, the method includes an initial phase, which may be an aggressive phase to rapidly lower blood sugars. In some embodiments, the method includes a maintenance phase. In some embodiments, the method includes a transition phase between an initial phase and a maintenance phase. In some embodiments, the method includes a hypoglycemic phase. In some embodiments, entering a hypoglycemic phase can affect future decisions (e.g., providing less insulin to reduce the chance of entering a hypoglycemic phase a second time). In some embodiments, the method uses an adaptive algorithm that responds to a patient's insulin sensitivity and adjusts infusion rates accordingly. The method may facilitate rapid correction in insulin infusion rates based on specific metabolic needs. This makes the method appropriate for critical care patients.

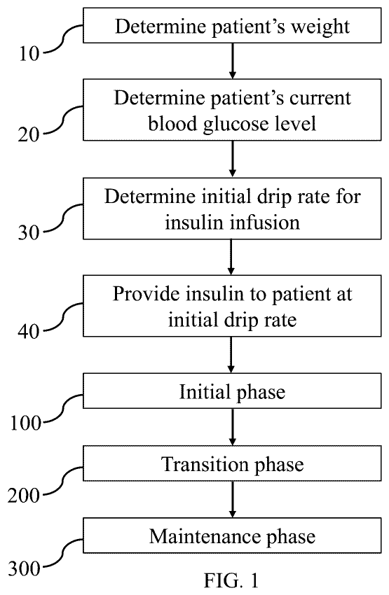

The schematic illustration in FIG. 1 shows a method of insulin infusion according to some embodiments. In some embodiments, the method is a computerized method for controlling insulin infusion. In some embodiments, the method utilizes a proportional-integral-derivative (PID) based dosing calculator to determine dosing rates (also referred to as drip rates). The PID calculator may rely on multiple glucose values and prior responses to insulin to determine insulin dosing rates. The method may be used to help patients that exhibit hyperglycemia during critical illness to control their blood sugars more safely and effectively. In some embodiments, the insulin is provided intravenously. Before insulin is administered to the patient, in some embodiments, data about the patient may be obtained. In some embodiments, the amount of patient data is kept to a minimum to expedite starting the insulin infusion process.

For example, in some embodiments, the patient's weight may be determined (operation 10). In some embodiments, the patient's height may also be determined. The patient's height and weight may be used to determine the patient's body mass index. In some embodiments, the patient's height and/or weight may already be known (e.g., from previous doctor visits). In some embodiments, the patient's current blood glucose value is also determined (operation 20). For example, a point-of-care blood glucose meter may be used to measure the patient's current blood glucose value.

In some embodiments, the patient's weight (and/or body mass index) and current blood glucose value are used at operation 30 to determine the initial drip rate for insulin infusion. For example, the blood glucose value may influence how the initial drip rate is calculated. In some embodiments, if the current blood glucose value is less than 80 mg/dL (which indicates hypoglycemia), the initial drip rate of insulin is 0 units/hour. In some embodiments, if the current blood glucose value is between 80 and 90 mg/dL, the initial drip rate is 0.25 units/hour. In some embodiments, if the current blood glucose value is between 91 and 100 mg/dL, the initial drip rate is 0.0125 times the patient's weight in kilograms. In some embodiments, if the current blood glucose value is between 101 and 150 mg/dL, the initial drip rate is 0.025 times the patient's weight in kilograms. In some embodiments, if the current blood glucose value is between 151 and 200 mg/dL, the initial drip rate is 0.0375 times the patient's weight in kilograms. In some embodiments, if the current blood glucose value is between 201 and 250 mg/dL, the initial drip rate is 0.05 times the patient's weight in kilograms. In some embodiments, if the current blood glucose value is between 251 and 300 mg/dL, the initial drip rate is 0.075 times the patient's weight in kilograms. In some embodiments, if the current blood glucose value is between 301 and 550 mg/dL, the initial drip rate is 0.1 times the patient's weight in kilograms.

In some embodiments, if the current blood glucose value is between 551 and 800 mg/dL, the initial drip rate may be 0.075 times the patient's weight in kilograms. In some embodiments, if the current blood glucose value is between 801 and 1000 mg/dL, the initial drip rate may be 0.05 times the patient's weight in kilograms. In some embodiments, if the current blood glucose value is between 1001 and 1500 mg/dL, the initial drip rate may be 0.025 times the patient's weight in kilograms. In some embodiments, if the current blood glucose value is above 1500 mg/dL, the initial drip rate may be 0.01 times the patient's weight in kilograms. In some embodiments, if the patient is in a hyperosmolar state and the current blood glucose value is 551 mg/dL or higher, the drip rate remains constant until the patient is no longer in a hyperosmolar state (e.g., it is not updated based on a new blood glucose reading unless the patient is no longer in a hyperosmolar state).

In some embodiments, a maximum drip rate may be used throughout the insulin infusion process. The maximum drip rate may be equal to 0.2 times the patient weight in kilograms in most situations. In some embodiments, the maximum drip rate is equal to 0.3 times the patient's weight in kilograms if the current glucose value is greater than 300 mg/dL and the glucose level has increased by more than 10% at least three times in the previous four hours. The initial drip rate may be checked against the maximum drip rate to ensure that the initial drip rate does not exceed the maximum drip rate. In some embodiments, the drip rates that are determined (e.g., maximum drip rates, initial drip rates, or other drip rates in any phase of the method) may be truncated after they are calculated. For example, in some embodiments, the drip rates are truncated to one decimal digit format (e.g., XX.Y). For example, if the calculated drip rate is 7.78, the administered drip rate would be truncated or rounded down to 7.7.

At operation 40, insulin is provided to the patient at the initial drip rate. Once the patient begins receiving insulin, the method is configured to quickly adapt to each patient to determine the optimal rate to achieve its goal of euglycemia. The recommendations for the optimal rate are computed in three different phases: an initial phase 100, a transition phase 200, and a maintenance phase 300. In some embodiments, there is also a hypoglycemic phase, in which the drip rate is set to 0.

The first phase (initial phase 100) is an aggressive phase during which blood sugars are rapidly lowered to achieve near normal blood sugar levels as fast as possible, keeping in mind that the osmolality changes need to be tolerated by the patient. Once blood sugar levels decrease towards euglycemia (e.g., by reaching a predetermined blood glucose value), the method changes to a less aggressive phase (transition phase 200) to assure a safe landing (transition) to the maintenance phase 300 (e.g., by avoiding lowering the blood sugars so much that the patient experiences a hypoglycemic event). The method achieves this safe transition by using different (dynamic) timed targets during the transition phase 200 so as to prevent hypoglycemia occurrence. Most of the hypoglycemia risk is during the period from the initial phase 100 to the maintenance phase 300.

### Initial Phase 100

As already noted, initial phase 100 is an aggressive phase in lowering blood sugars rapidly. To facilitate this aggressive phase, the initial drip rate (and subsequent drip rates during initial phase 100) may be calculated that help quickly lower blood sugars. In some embodiments, the initial phase 100 relies on recent insulin drip rates and blood glucose levels to determine the patient's insulin sensitivity and adjust the insulin infusion rate accordingly. Initial phase 100 continues until the patient's blood glucose level reaches a predetermined blood glucose value. In some embodiments, the predetermined blood glucose value is between about 180 and about 220 mg/dL (though other values may be used as well). In some embodiments, the predetermined blood glucose value is between 190 and 210 mg/dL. In some embodiments, the predetermined blood glucose value is 200 mg/dL.

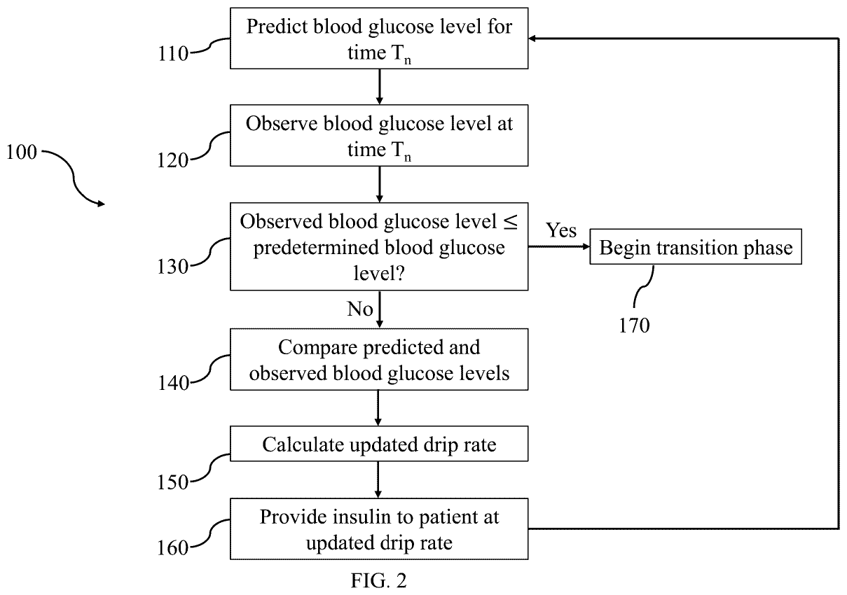

An example approach for initial phase 100 is shown in FIG. 2 according to some embodiments. In some embodiments, administering insulin at the initial drip rate (operation 40) may be considered to be part of initial phase 100. In some embodiments, at operation 110, the blood glucose level for a certain point in time T₁ is predicted (represented by Tₙ in FIG. 2). This prediction may be based on the initial drip rate and the starting blood glucose level of the patient. In some embodiments, the predicted blood glucose level is 85% of the starting blood glucose level. In some embodiments, the certain point in time T₁ may be anywhere from 30 minutes to 2 hours (e.g., 1 hour) after the insulin is first provided to the patient. In some embodiments, the certain point in time T₁ may be selected by a user (e.g., a healthcare provider). In some embodiments, the certain point in time T₁ is fixed such that a user cannot modify it.

In some embodiments, at operation 120, once the certain point in time T₁ has arrived, the blood glucose level of the patient is observed at T₁. This may be done using a point-of-care blood glucose meter, a continuous glucose monitor, or some other device to measure blood glucose level. At operation 130, the observed blood glucose level is compared with the predetermined blood glucose level (e.g., 200 mg/dL). If the observed blood glucose level is equal to or less than the predetermined blood glucose level, the initial phase 100 is complete and the transition phase 200 begins (at operation 170). If the observed blood glucose level has not reached the predetermined blood glucose level, the initial phase 100 continues at operation 140. In some embodiments, at operation 140, the predicted blood glucose level (from operation 110) and the observed blood glucose level (from operation 120) are compared with each other.

In some embodiments, at operation 150, an updated drip rate is calculated. The updated drip rate may be calculated based on the difference between predicted and observed blood glucose levels (such as the calculations discussed in more detail below). For example, if the observed blood glucose level is higher than the predicted blood glucose level, the updated drip rate may be higher than the initial drip rate (or the previous drip rate). If the observed blood glucose level is lower than the predicted blood glucose level, the updated drip rate may be lower than the initial drip rate (or the previous drip rate). In some embodiments, this method is configured to identify the patient's insulin sensitivity, as well as patterns, over time based on how the observed blood glucose level compares to the predicted blood glucose level. This may allow future predicted blood glucose levels to be more accurate, which may result in the drip rate being more effective at reaching the target blood glucose level more quickly.

In some embodiments, the updated drip rate is calculated based on recent trends of blood glucose level, in addition to the difference between predicted and observed blood glucose levels. The recent trends (for example, how quickly the blood glucose level changes in response to insulin) can be determined by a lookback period, in which blood glucose levels and targets (e.g., predicted levels) at any particular times from previous points in time may be evaluated. In some embodiments, throughout the initial phase 100, calculating the next drip rate depends on the difference between the predicted and observed blood glucose levels and the trend (e.g., the lookback period) of the blood glucose level in the previous hours and the interim target blood glucose values at any particular time.

“Lookback period” means a defined retrospective time window or sequence of consecutive blood glucose measurements used to evaluate directional trends and cumulative changes in a patient's blood glucose measurements for drip-rate adjustment logic. In some embodiments, the lookback period may include four consecutive readings (a “four-period period”) spanning about 30 minutes to 4 hours. During this time, the system determines whether each successive blood glucose measurement moved in the same direction (i.e., all increasing or all decreasing) and examines the cumulative magnitude of change across those readings. The lookback period enables detection of sustained trends rather than isolated fluctuations and serves as a trigger condition for increasing or decreasing the insulin infusion rate during the maintenance phase.

In some embodiments, at operation 160, insulin is provided to the patient at the updated drip rate. Initial phase 100 continues by returning to operation 110. The blood glucose level for time Tₙ may be predicted based on the updated drip rate and the most recent observed blood glucose level. In some embodiments, the certain point in time Tₙ may be anywhere from 30 minutes to 2 hours after the insulin is provided to the patient at the updated drip rate. For example, the method may predict (and then observe) a blood glucose level at hourly intervals. The amount of time between certain points in time Tₙ may be equal to each other, or they may vary depending on the current blood glucose level and/or the rate of change of the blood glucose level.

Initial phase 100 continues until the observed blood glucose level is equal to or less than the predetermined blood glucose level, at which point the process proceeds to transition phase 200, as shown at operation 170.

In some embodiments, the amount of time for initial phase 100 is predetermined. This predetermined amount of time may be used to work backwards to alter the drip rates so that the time to euglycemia is kept constant. For example, the drip rates may be calculated to have a fixed 4-hour initial phase 100 so that the blood glucose level reaches near euglycemia (i.e., the predetermined blood glucose level (e.g., 200 mg/dL)). In some embodiments, this approach uses an aggressive drip rate as needed, especially when initial blood glucose levels are between 300 mg/dL and 600 mg/dL. This may be particularly useful where time is of the essence, such as for those undergoing emergency surgery. In some embodiments, a 3-hour initial phase may be used.

In some embodiments, this approach is only allowed as long as there is no evidence of a hyperosmolar state, in which caution is observed during the initial phase to prevent neurological damage and complications. In some embodiments, another prerequisite for this approach is that electrolytes need to be in normal range or at least simultaneously corrected as needed. In some embodiments, an osmolar guide that restricts changes in osmolality exerted by glucose decreases (e.g., target range of fall is 3-6 mosmols/L) restricts fall in blood glucose levels to prevent neurological damage or complications. The osmolar guide may be most useful in hyperosmolar states.

For example, in some embodiments, if the patient's sodium is above 145 when the patient is in a hyperosmolar state, then the drop in blood sugars may be limited to 50 mg/dL per hour (which is about a 2.78 drop in osmolality). In some embodiments, if the patient's sodium is lower than 145 when the patient is in a hyperosmolar state, then the drop in blood sugars may be limited to 100 mg/dL per hour (which is about a 5.55 drop in osmolality).

In some embodiments, if sodium is below 130 caution may be exercised. In some embodiments, if the initial blood sugar is less than 650 mg/dL, then the method may proceed with drip rates (as described above), along with fluid resuscitation as clinically determined. On the other hand, if the initial blood sugar is greater than 650 mg/dL, then the serum sodium and/or urine sodium may be checked to determine sodium loss, which should caution the initiation of insulin drip. Instead, fluid resuscitation may be initiated and continued until blood sugar falls below 650 mg/dL, after which insulin drip can be started with a target drop in blood sugars of 5-10% per hour. If there are no changes in serum sodium and blood sugars are between 650 and 800 mg/dL, then insulin drip and fluid resuscitation may be initiated together, but the target drop in blood sugars should be 10-15% per hour until the glucose level falls below 650 mg/dL. If blood sugars are above 800 mg/dL, the initial step is to use only fluid resuscitation.

In some embodiments, the osmolality is checked. If the osmolality is normal (between 275 and 295 mOsm/kg), the process may proceed as discussed above. If the osmolality is above 305 mOsm/kg, caution is exercised, but the process may proceed. However, if the osmolality is above 320, no insulin is provided until osmolality falls below 305 mOsm/kg.

If the blood sugar is greater than 650 mg/dL and serum sodium is below normal, then fluids may be used with caution to treat severe dehydration, keeping in mind the total sodium loss and the time to normalization using the formula of sodium resuscitation. In some embodiments, until the blood sugar falls below 650 mg/dL or reaches a steady state (within +/−5% for four hours) above 650 mg/dL, the starting drip rate may be set at 0.05 times the patient weight (in kilograms), with a target drop rate of 5-10% every hour.

### Transition Phase 200

As already noted, transition phase 200 is a less aggressive phase than initial phase 100. The transition phase 200 allows a patient to reach euglycemia while reducing the risk overshooting euglycemia and experiencing hypoglycemic events. In some embodiments, transition phase 200 follows a similar process as initial phase 100. However, the drip rates are calculated in a way that accomplishes a more gradual change in blood glucose level.

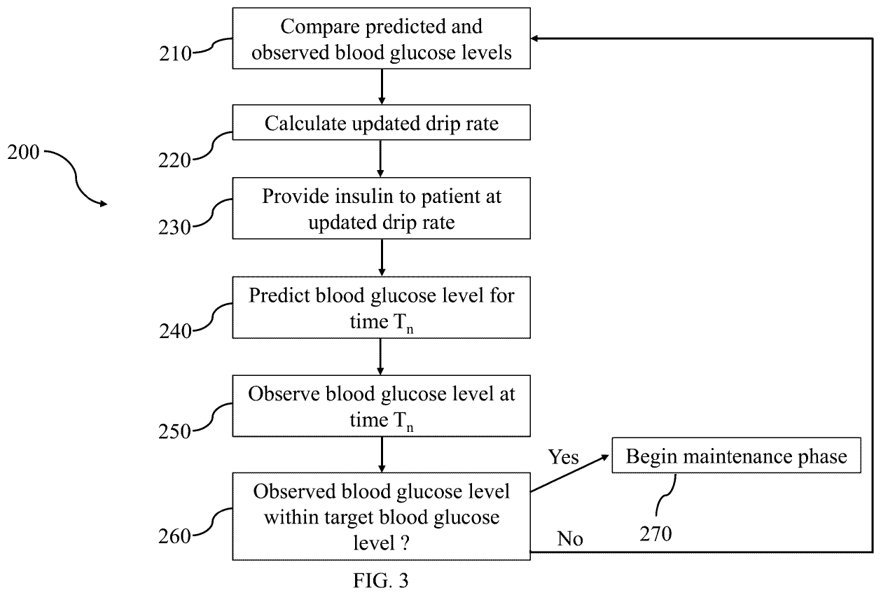

An example approach for transition phase 200 is shown in FIG. 3 according to some embodiments. Operation 210 may immediately follow operations 130 and 170. In some embodiments, at operation the predicted blood glucose level (from the most recent operation 110 in initial phase 100) and the observed blood glucose level (from the most recent operation 120 in initial phase 100) are compared with each other.

In some embodiments, at operation 220, an updated drip rate is calculated. The updated drip rate may be calculated based on the difference between predicted and observed blood glucose levels and/or recent trends. This calculation may be similar to the calculation of drip rates at operation 150 in initial phase 100, except that the calculated drip rate seeks to achieve a slower change in blood glucose level. In some embodiments, throughout the transition phase 200, calculating the next drip rate depends on the difference between the predicted and observed blood glucose levels and the trend (e.g., the lookback period) of the blood glucose level in the previous hours and the interim target blood glucose values at any particular time.

In some embodiments, at operation 230, insulin is provided to the patient at the updated drip rate. In some embodiments, at operation 240, the blood glucose level for time Tₙ may be predicted based on the updated drip rate and the most recent observed blood glucose level. In some embodiments, the certain point in time Tₙ may be anywhere from 30 minutes to 2 hours after the insulin is provided to the patient at the updated drip rate. For example, the method may predict (and then observe) a blood glucose level at hourly intervals. The amount of time between certain points in time Tₙ may be equal to each other, or they may vary depending on the current blood glucose level and/or the rate of change of the blood glucose level.

In some embodiments, at operation 250, once the certain point in time Tₙ has arrived, the blood glucose level of the patient is observed at Tₙ. This may be done using a point-of-care blood glucose meter, a continuous glucose monitor, or some other device to measure blood glucose level.

In some embodiments, at operation 260, it is determined whether the observed blood glucose level is within the target blood glucose level. In some embodiments, the target blood glucose level is between 70-180 mg/dL. In some embodiments, the target blood glucose level is between 100-140 mg/dL. In some embodiments, the target blood glucose level is 120 mg/dL. The target blood glucose level may be any value within euglycemia, a narrower range within euglycemia, or a specific value within euglycemia. If the observed blood glucose level is within the target blood glucose level, the transition phase 200 is complete and the maintenance phase 300 begins (at operation 270). If the observed blood glucose level has not reached the target blood glucose level, the transition phase 200 continues by returning to operation 210. The most recent predicted blood glucose level (from operation 240) and the most recent observed blood glucose level (from operation 250) are compared with each other, after which an updated drip rate is calculated at 220 and so on. Transition phase 200 continues until the observed blood glucose level is within the target blood glucose level, at which point the process proceeds to maintenance phase 300, as shown at operation 270.

### Maintenance Phase 300

In some embodiments, maintenance phase 300 operates using a proportional-integral-derivative (PID) based dosing calculator to determine dosing rates. The PID calculator may rely on multiple glucose values and prior responses to insulin to determine insulin dosing rates. In some embodiments, the PID calculator learns patterns over time to calculate dose rates that are more accurate in achieving the desired blood glucose level.

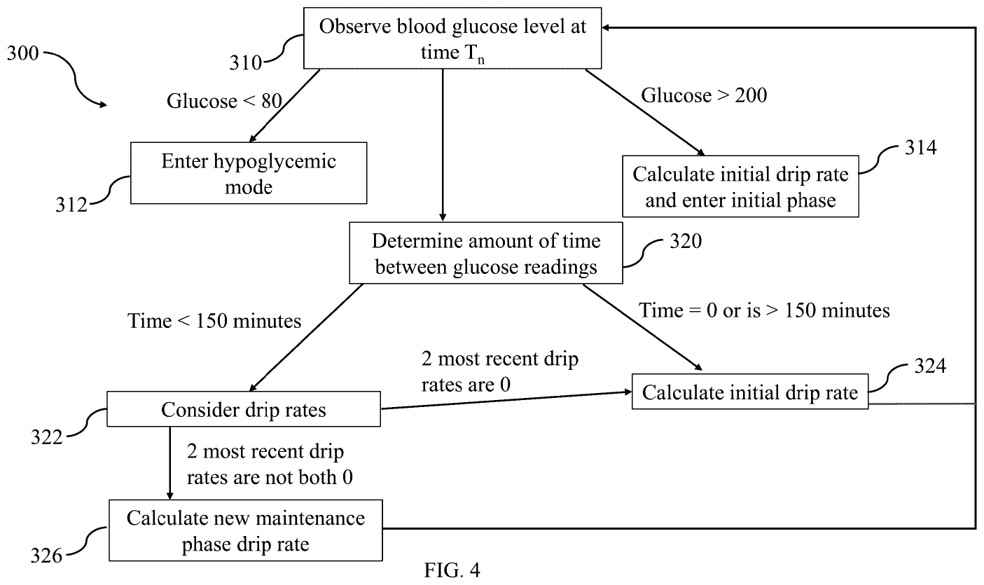

An example maintenance phase 300 is shown in FIG. 4. In some embodiments, during maintenance phase 300, regular glucose readings are taken (e.g., every hour). For example, at operation 310, the blood glucose level of the patient is observed at Tₙ. This may be done using a point-of-care blood glucose meter, a continuous glucose monitor, or some other device to measure blood glucose level. If the blood glucose level is less than 80 mg/dL, then at operation 312, the method enters a hypoglycemic mode, in which the drip rate is set to 0. If the blood glucose level is greater than 200 mg/dL, then the method enters initial phase 100 at operation 314. In some embodiments, an initial drip rate may be calculated based on the glucose level, for example, using a table as discussed above, as part of operation 314.

If the glucose level is between 80 and 200 mg/dL, then at operation 320, the amount of time between glucose readings is determined. In some embodiments, if the current drip rate (at the time of observing the blood glucose level in operation 310) is 0 units per hour, then the glucose reading time from the entry prior to the 0 units per hour entry is obtained. Otherwise, the time of observing the blood glucose level in operation 310 is set as the last glucose reading.

If there are less than 150 minutes between the current glucose value and the last glucose value time, the method continues at operation 322 by considering the drip rates (e.g., the current drip rate and the previous drip rate). If the two most recent drip rates are both 0 units per hour, then the method continues at operation 324 by calculating an initial drip rate (using a table as discussed above). This initial drip rate may then be applied to the patient for another period of time (e.g., 1 hour) at which point the method continues with operation 310. On the other hand, if the two most recent drip rates are not both 0 units per hour, a new maintenance phase drip rate is calculated at operation 326. For example, if the current drip rate is 0 units per hour, and the previous drip rate is greater than 0, then the new drip rate is calculated to be half of the previous drip rate (i.e., half of the drip rate before the 0 units per hour drip rate). However, if half of the drip rate is less than 0.5, then the method may move to operation 324 and calculate an initial drip rate based on a table as discussed above. If the current drip rate is not 0 units per hour, the new drip rate may be calculated in one of two ways. If the current glucose level is less than 100 mg/dL and the last glucose level was greater than 100 mg/dL, then the new drip rate is calculated to be half of the current drip rate. Otherwise, the new drip rate is calculated by adding the current drip rate and the product of the percentage change in glucose and the current drip rate (New drip rate=current drip rate+(% change in glucose*current drip rate)).

If the determined amount of time at operation 320 is equal to 0 or is greater than 150 minutes, then the method proceeds to operation 324 and an initial drip rate is calculated based on a table as discussed above.

During the maintenance phase 300, the goal is to maintain euglycemia while preventing hyperglycemic and/or hypoglycemic surges. A lot of factors, including meals consumed, new medications, state and stress of the illness, etc., can affect blood glucose levels, warranting a change in the insulin drip rates during this maintenance phase 300. With these factors at play, in some instances, the blood glucose level may become unstable. In some embodiments, the blood glucose level is considered unstable if there is more than a 15% change in blood glucose level within a one-hour interval. Thus, in some embodiments, the method constantly looks back to recognize changes in the blood glucose levels and trends over a predetermined lookback period to evaluate and prevent large excursions in blood glucose levels. In some embodiments, the predetermined lookback period is between 30 minutes and 4 hours. A longer lookback period may help identify trends and patterns. This lookback period may help prevent loss of control of blood glucose level once control has been achieved.

In some embodiments, during the lookback period in maintenance phase 300, the method may look for multiple readings that are moving in the same direction (e.g., each consecutive reading is decreasing in glucose value). If the cumulative magnitude of change from these readings is more than a 10% decrease within the previous 2-4 hours, then the drip rate may be decreased by the same average percentage. In some embodiments, if the blood glucose level is less than 100 mg/dL and there is a 10% reduction in one hour, then the drip rate may be reduced by 50%. In some embodiments, if the blood glucose level is less than 100 mg/dL and there is a 15% reduction in one hour, then the drip rate may be reduced by 75%. In some embodiments, if the blood glucose level is more than 130 mg/dL and the cumulative magnitude of change from multiple readings is more than a 15% increase within the previous 2-4 hours, then the drip rate may be increased by the same average percentage. In some embodiments, the cumulative magnitude may be compared to a predetermined magnitude. In some embodiments, a “predetermined magnitude” is a configurable threshold amount of the cumulative change in blood glucose level, expressed as a percentage over the lookback period, that must be exceeded before the system permits a drip-rate adjustment. In some embodiments, the system may include additional criteria, such as those discussed throughout the present disclosure, before the system permits a drip-rate adjustment.

In some embodiments, the lookback period facilitates identifying patterns, especially with the use of artificial intelligence. For example, factors such as medications, illnesses, food, etc. can become better understood for the particular patient through pattern recognition. As one example, over time, the method may identify how a patient's insulin sensitivity changes with a particular sickness, or a particular medication, and thus how the patient's blood glucose level may change in response to the particular sickness or medication. The method can then account for these known factors in determining the dosing rates for insulin to prevent excessive changes in blood glucose level. Thus, the method is adaptive and becomes more accurate over time.

In some embodiments, during maintenance phase 300, blood glucose levels are measured at one-hour intervals. In some embodiments, other intervals may also be used. Moreover, in some embodiments, the interval times may be changed (e.g., by a user, based on measured glucose values, history of glucose values, trends, etc.). In some embodiments, the method may have the capacity to accommodate changes in the interval times in its calculation. For example, there may be times when the glucose is not measured in exact 1 hour increments. In some embodiments, the method measures the elapsed time between readings in minutes (rather than assuming an exact hour has passed) and accounts for the actual number of minutes elapsed in its calculations.

In some embodiments, a continuous glucose monitor (CGM) may be used to measure blood glucose levels (instead of or in addition to a point-of-care blood glucose meter). In some embodiments, when a CGM is used to determine data points to determine drip rates, an average may be used for the observed blood glucose level. For example, an average of blood glucose levels within fifteen-minute intervals (or smaller intervals) may be adopted, and then an average of these averages (e.g., over about a one-hour interval) may be computed. In some embodiments, the latest values may be given a greater weightage in determining the overall average so that the latest data has a greater effect on the determined average than earlier values.

In some embodiments, when using CGM data, a point-of-care blood glucose meter is used in one-hour intervals during the initial phase 100 and transition phase 200 to corroborate with the blood glucose levels obtained via the CGM. In some embodiments, during maintenance phase 300, a point-of-care blood glucose meter is used every four to six hours to corroborate with the blood glucose levels obtained via the CGM.

In some embodiments, when using a CGM, every hour the blood glucose value is assessed for any change. For example, the data for that hour may be assessed (e.g., the fifteen-minute intervals data (or smaller intervals)). If a drop is noted that is consistent, this is treated as a drop similar to non-CGM applications. However, if the values are inconsistent or erratic, different approaches may be taken. If the values move up and down, but they are higher at the end of the hour, the drip rate may be increased according to the regular formulas. If the values move up and down, but they are lower at the end of the hour, the drip rate may be decreased according to the regular formula. If the values are changing faster towards the end of the hour (e.g., the more recent 15 minute intervals), then the change in drip rate may be multiplied by a factor (e.g., 1.5×). If hypoglycemia is determined, the drip rate may be decreased at a shorter interval than one hour.

In some embodiments, towards the end of the IV infusion (i.e., the end of maintenance phase 300) the method determines the current insulin requirements and the patient's sensitivity to insulin. In some embodiments, the method then compares the current insulin requirements and the patient's sensitivity to a weight-based formula to compute a suggested basal insulin dosage (e.g., for after the IV infusion). In some embodiments, the basal insulin dosage is provided before ending the IV infusion. For example, the basal insulin dosage may be first administered subcutaneously two hours before ending the IV infusion, thus providing a two-hour overlap period.

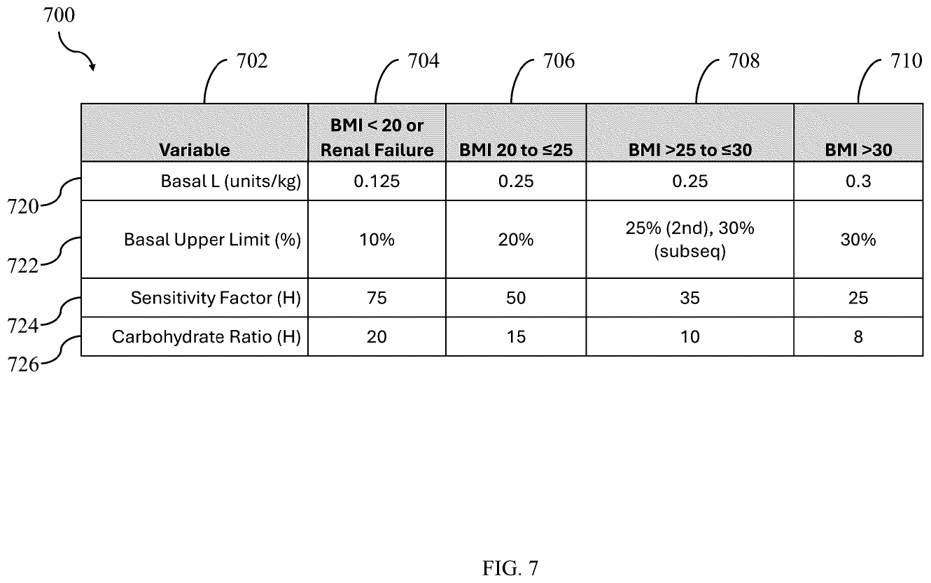

In some embodiments, the weight-based formula is dependent on the current phase and whether a lookback period triggered a change (for example, is +/−15% deviation). For example, if the method is in the maintenance phase and no lookback period has been triggered, then the weight-based formula for basal insulin may be equal to 0.25 times the weight of the patient in kilograms. In this situation, the sum of drip rates from the last 8 hours is considered the observed insulin dosage. If the observed insulin dosage is greater than the weight-based formula, then the midpoint between the observed insulin dosage and the weight-based formula is used. If the observed insulin dosage is less than the weight-based formula, then the observed insulin dosage is used for the basal insulin dosage. In some embodiments, the weight-based multiplier is determined with reference to FIG. 7's table.

In all other situations (the method is either not in the maintenance phase or a lookback has triggered a change), then the following formula is used to determine basal insulin dosage. The previous four readings are then considered for determining the observed insulin dosing rate (L0 is the most recent reading, L1 is the reading 1 hour prior, L2 is the reading 2 hours prior, and L3 is the reading 3 hours prior). A weighted hourly rate is determined using the following formula: ((4*L0)+(3*L1)+(2*L2)+(L3))/10. The observed insulin dosage is calculated as 8 times the weighted hourly rate. If the observed insulin dosage is greater than the weight-based formula, then the midpoint between the observed insulin dosage and the weight-based formula is used. If the observed insulin dosage is less than the weight-based formula, then the observed insulin dosage is used for the basal insulin dosage.

In some embodiments, the disclosed method uses a PID (proportional-integral-derivative) controller to achieve greater accuracy. In some embodiments, the method uses dynamic targets over time to achieve euglycemia rapidly during the initial phase 100, thus reducing errors all along the way to ensure accuracy. For example, the initial phase 100 may have an hourly target of a 15% reduction in blood glucose value.

The adaptive algorithm continuously evaluates patient-specific data, including real-time glucose readings and historical response trends, to adjust insulin infusion rates. It incorporates a proportional-integral-derivative (PID) control model that dynamically recalibrates dosing based on deviation from predicted glucose levels, rate of change, and cumulative error over a lookback period.

In some embodiments, the disclosed method provides an improved clinical workflow (e.g., reduced burden in starting the insulin infusion, with adjustments being made based on a computerized method).

In some embodiments, the method may include fewer or more steps than outlined above. For example, in some embodiments, a new start or initial action may begin as described above (determining an initial first rate using a table based on current glucose level and patient weight). In some embodiments, an initial drip mode may also be determined. If the glucose level is less than 80 mg/dL, then the initial drip mode is hypoglycemic mode. If the glucose level is between 80 and 135 mg/dL, then the initial drip mode is maintenance mode. If the glucose level is above 135 mg/dL, then the initial drip mode is an induction mode. After determining the initial drip rate and drip mode, new glucose readings are taken periodically (e.g., every hour). The method may then follow a decision tree in response to each new glucose reading based on the last documented drip mode prior to the reading.

If currently in the hypoglycemic mode, the drip rate should already be set to 0 units per hour. The drip rate may stay at 0 until the new glucose reading. If the new glucose reading is less than 80 mg/dL, the method remains in the hypoglycemic mode. If the new glucose reading is greater than or equal to 80 and less than or equal to 200 mg/dL, then the method enters the maintenance mode and then calculates a drip rate (using the maintenance mode criteria). If the new glucose reading is greater than 200 mg/dL, then the method enters the induction mode and then calculates a drip rate (using the induction mode criteria).

In some embodiments, if currently in the maintenance mode, the following decision tree may be used.
1. If the glucose reading is less than 80 mg/dL, the method enters the hypoglycemic mode, which sets the drip rate to 0.
2. If the glucose reading is greater than 200 mg/dL, the method enters the induction mode and then calculates a drip rate (using the induction mode criteria).
3. If the current drip rate is 0 units per hour, then the glucose reading time from the entry prior to the 0 units per hour is obtained. Otherwise, the time of observing the blood glucose level is set as the last glucose reading.
4. If there are less than 150 minutes between the current glucose value and the last glucose value time then the following logic is used.
4a. If the current and prior drip rates are both 0 units per hour, then the method restarts with the initial logic (the initial action mentioned above).
4b. If the current drip rate is 0 units per hour and the prior drip rate is greater than 0 units per hour, then the new drip rate is calculated as half of the drip rate prior to the 0 units per hour drip rate. If this calculation is less than 0.5, then the method restarts with the initial logic.
4c. If the current drip rate is not 0 units per hour, and if the current glucose level is less than 100 mg/dL and the last glucose level was greater than 100 mg/dL, the current drip rate is cut in half. If the current drip rate is not 0 units per hour, and the other condition in the previous sentence (i.e., current glucose level less than 100 mg/dL and the previous glucose level greater than 100 mg/dL) is not met, the new drip rate is calculated by adding the current drip rate and the product of the percentage change in glucose and the current drip rate (New drip rate=current drip rate+(% change in glucose*current drip rate)).
5. If there were either 0 or more than or equal to 150 minutes between glucose values, then the method restarts with the initial logic.

If currently in the induction mode, the following decision tree is used.
1. If the previous glucose value is greater than or equal to 800 mg/dL and the current glucose reading is greater than or equal to 150 mg/dL and there is greater than or equal to a 250 mg/dL decrease in glucose between the two values, then the current drip rate is decreased by 25%.
2. If there is greater than or equal to a 30% decrease in glucose form the previous value to the current value, the following logic is used:
2a. If the current glucose is less than 80 mg/dL, the drip mode is set to the hypoglycemic mode and the drip rate is set to 0 units per hour.
2b. If the current glucose is between 80 and 120 mg/dL, the drip mode is set to the maintenance mode and the drip rate is calculated by decreasing the current rate by 75%.
2c. If the current glucose is greater than or equal to 120 mg/dL and less than or equal to 150 mg/dL, the drip mode is set to the maintenance mode. If the glucose decreased more than or equal to 30% and less than 40% from the previous value, then the drip rate is decreased by 60%. If the glucose decreased more than or equal to 40% and less than 50% from the previous value, then the drip rate is decreased by 70%. If the glucose decreased more than or equal to 50% from the previous value, then the drip rate is decreased by 75%.
3. If the conditions in 1 and 2 (including the additional conditions in 2a, 2b, or 2c) above are not met, then the following logic is used.
3a. If the current glucose is less than 80 mg/dL, the drip mode is set to the hypoglycemic mode and the drip rate is set to 0 units per hour.
3b. If the current glucose is between 80 and 120 mg/dL, the drip mode is set to the maintenance mode. In addition, the following logic is used.
3b1. If the previous drip rate is 0 units per hour and the current glucose is 80-90 mg/dL, then the drip rate is set to 0.25 units per hour. If the previous drip rate is 0 units per hour and the current glucose is 91-100, then the drip rate is set to 0.0125 times the patient weight (in kilograms). If the previous drip rate is 0 units per hour and the current glucose is 101-120, then the drip rate is 0.025 times the patient weight (in kilograms).
3b2. If the previous drip rate is greater than 0 and less than or equal to 2 units per hour, then the drip rate is set at 0.25 units per hour.
3b3. If the previous drip rate is greater than 2 units per hour, then the drip rate is decreased by 50% of the current rate.
3c. If the current glucose is greater than or equal to 120 mg/dL, then the following logic is used.
3c1. If the previous drip rate is 0 units per hour and the drip rate prior to that is also 0, then the method restarts with the initial logic.
3c2. If the current drip rate is 0 units per hour, then the glucose reading time from the entry prior to the 0 units per hour is obtained. Otherwise, the time of observing the blood glucose level is set as the last glucose reading.
3c3. If 150 minutes or more have elapsed between the current glucose value and the last glucose value time, then the method restarts with the initial logic.
3c4. If less than 150 minutes (and more than 0) have elapsed between the current glucose value and the last glucose value time, then the following logic is used.
3c4a. If the previous drip rate is 0 units per hour, then the drip rate is set to half of the prior drip rate that is not zero. However, if this results in the drip rate being less than 0.5 units per hour then the method restarts with the initial logic.
3c4b. If the previous drip rate is not zero, then the ratio of current glucose value to the desired glucose value is calculated. The desired glucose value is defined as 85% of the previous glucose value (which targets a 15% reduction). The 15% reduction target may be based on measurements obtained every hour. However, the blood glucose measurement may not be done exactly at hour intervals. The method can account for this variation by basing its calculations on the time between glucose readings in minutes. For example, the ratio may be calculated with the following equation: Ratio=current blood glucose level/(previous glucose level*(0.85\hat{ }(time between glucose readings in minutes/60))).
3c4c. Factors are determined for later use in calculating a new drip rate. If the current glucose value is less than 190 mg/dL, then a “LessFactor” is set to 0.05 and a “MoreFactor” is set to 0.1. If the current glucose value is greater than or equal to 190 mg/dL, then a “LessFactor” is set to 0.1 and a “MoreFactor”is set to 0.05.
3c4d. If the Ratio (from 3c4b above) is between 0.95 and 1.06, the current blood glucose level is within a 6% deviation from the target. In this situation, there may be no change in drip rate. Thus, the drip rate may be set to the previous drip rate.
3c4e. If the Ratio (from 3c4b above) is greater than 1.06, then the glucose decreased less than expected. As a result, the drip rate may be increased. The variation may be calculated as equal to (Ratio-1.05)*100. In this situation, the drip rate may be calculated as equal to the previous drip rate added to the product of the variation (as calculated in the previous sentence), the previous drip rate, and the LessFactor. If this calculated drip rate is greater than twice the previous drip rate, then the drip rate may be set to twice the previous rate. Otherwise, the drip rate is set to the calculated drip rate.
3c4f. If the Ratio (from 3c4b above) is less than 0.95, then the glucose decreased more than expected. As a result, the drip rate may be decreased. The variation may be calculated as equal to (0.95-Ratio)*100. In this situation, the drip rate may be calculated as equal to the previous drip rate minus the product of the variation (as calculated in the previous sentence), the previous drip rate, and the MoreFactor. If this calculated drip rate is less than half the previous drip rate, then the drip rate may be set to half the previous rate. Otherwise, the drip rate is set to the calculated drip rate.

### Insulin Infusion System

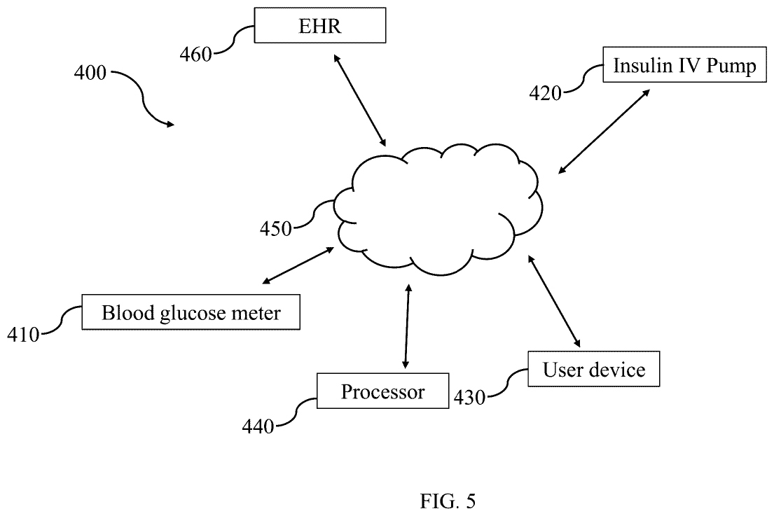

The present disclosure also provides a system of insulin infusion that may be used to implement the method disclosed herein. An example schematic illustration of a system 400 is shown in FIG. 5 according to some embodiments. In some embodiments, the system 400 includes a blood glucose meter 410, an insulin IV pump 420, a user device 430, a processor 440, a network 450, and an electronic health record (EHR) 460. One or more of these components may be omitted in some embodiments. In addition, one or more of these components may be combined into a single device.

In some embodiments, the components of system 400 may communicate with each other over network 450, which may be a wireless network or a wired network. In some embodiments, blood glucose meter 410 is used to take point-of-care blood glucose measurements. In some embodiments, insulin IV pump 420 is used to administer insulin to the patient. In some embodiments, a user may enter data into system 400 and/or control insulin IV pump 420 via user device 430. In some embodiments, processor 440 may be configured to perform the method described herein. For example, processor 440 may receive measurements of the blood glucose level from blood glucose meter 410 (e.g., over network 450, or by a user entering the measurement through user device 430) and use those measurements to calculate a dosing rate (as discussed above). In some embodiments, processor 440 may be configured to control insulin IV pump 420 based on the dosing rates that are determined. Alternatively, the dosing rates that are determined may be displayed on user device 430, and a user may control insulin IV pump 420 to administer insulin at the determined dosing rate.

In some embodiments, the system 400 allows for interaction with an electronic health record 460. For example, the insulin infusion system 400 may be in communication with the electronic health record 460 and may obtain data about a patient from the electronic health record 460 and/or provide data to the electronic health record 460 (e.g., information learned from the insulin infusion). In some embodiments, the system integrates directly into the electronic medication administration record of the electronic health record. This may streamline the data entry process, as data is only needed to be input in one place (e.g., via user device 430).

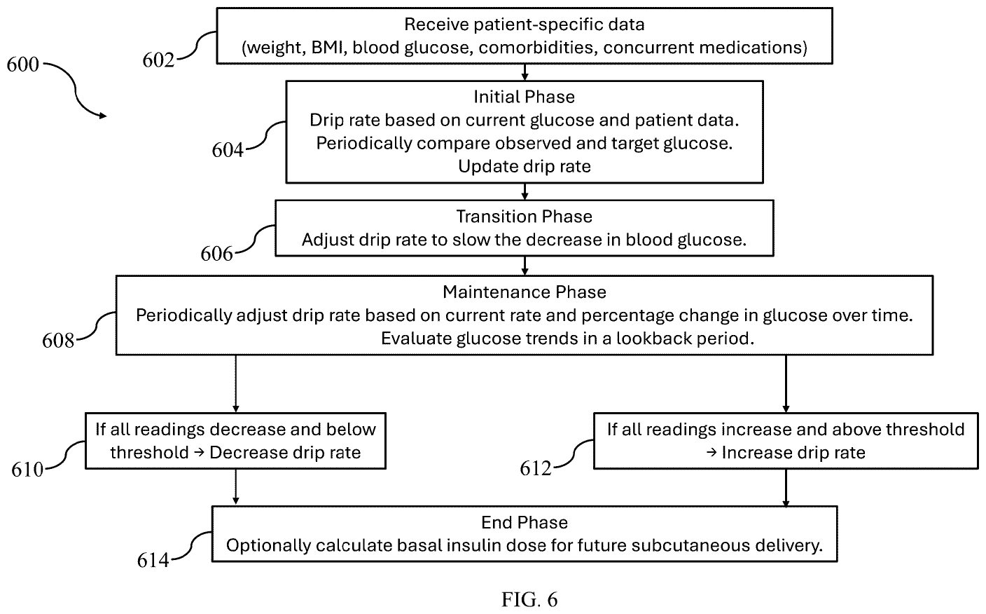

FIG. 6 shows method 600 of administering insulin to a patient to achieve and maintain euglycemia. In some embodiments, method 600 includes receiving patient-specific data 602. In some embodiments, the patient-specific data includes information about patient weight, BMI, blood glucose (for example, historic readings), comorbidities, and concurrent medications. Method 600 may also include administering insulin during an initial phase 604, a transition phase 606, and a maintenance phase 608. Method 600 may also evaluate readings during maintenance phase 608 and, if all readings decrease and are below a threshold, then method 600 may include decreasing the insulin drip rate 610. If all readings increase and are above a threshold, then method 600 may include increasing the insulin drip rate 612. In some embodiments, method 600 may include an end phase 614. End phase 614 may calculate a basal insulin does for future subcutaneous deliver to the patient.

A system for administering insulin to a patient to achieve and maintain euglycemia will now be described in detail with reference to FIG. 7-9. In some embodiments, system 900 is configured to output a basal dosing instruction to insulin IV pump 938. In some embodiments, system 900 is configured to determine a total daily dose (TDD) for a patient and sending instructions to administer the same to insulin IV pump 938. The TDD may change on a dynamic basis as the patient's needs change.

System 900 is communicatively coupled to a user device 932, a blood glucose meter 936, an electronic health records database (EHR) 934, and an insulin IV pump 938. User device 932 may be a computer, tablet, or other electronic device. The healthcare provider interfaces with system 900 through user device 932. In some embodiments, user device 932 is several devices. For example, user device 932 may be a bedside user device (PC workstation) and the healthcare provider's tablet. Both may display the same information and may be used to confirm or enter an order into system 900. In some embodiments, blood glucose meter 936 to take blood glucose measurement from the patient and report the measurement to system 900. Processor 902 may be configured to receive the blood glucose measurement from blood glucose meter 936. The readings provided by the blood glucose meter are used for adjusting insulin dosages to maintain euglycemia, which is the state of having normal blood glucose levels.

In some embodiments, the user device has a graphical user interface generated by the system. The graphical user interface may be organized into sections. Each section may provide different aspects of care information to the health care provider. For example, a first section may include the patient's biographic information (Name, Age, Sex) and the second section may include relevant portions of the patients medical history, or identify unknowns from the patient's medical history that need to be determined. In some embodiments, a third section may include information on the current treatment or proposed treatment plan, including target blood glucose levels, the administration type (basal only, or basal and bolus). Additional sections may include: protocol reports, key glycemic metrics, labs, instructions (including Action Items from nudges), action buttons, glucose levels over time, and osmolality levels over time.

System 900 may prompt user device 932 or query electronic health records (EHR) 934 to determine system 900 inputs. For example, processor 902 may query user device 932 to determine if the patient is on a known home dose. For example, this could prompt the user, the healthcare provider, to enter the total basal daily dose (TBDD) in units per day the patient was on at home. In some embodiments, only if TDD is known, system 900 may set TBDD at 50% of TDD. At another step, system 600 may prompt user device 932 or query electronic health records (EHR) 934 to determine if the patient has a history of hypoglycemia at home. If so, system 600 may set the TDD to an initial dose as shown in control table 700 shown in FIG. 7. If not, the initial dose will be the lower of 40% of the bodyweight or the TBDD.

FIG. 7 shows control table 700 according to some embodiments. Control table 700 presents initial values for different variables 702 used to calculate the initial TDD. FIG. 7 shows the following variables: Basal L (units/kg) 720, Basal Upper Limit (%) 722, Sensitivity Factor (H) 724, and the Carbohydrate Ratio (H) 726. Initial values for each variable 702 used to calculate the initial TDD are determined based on patient information (BMI and Renal Failure) shown in 704, 706, 708, and 710.

In some embodiments, control table 700 may dynamically update during patient care to change variables 702. For example, these values are updated by the method during patient care to tailor each to the patient's specific needs. For example, if the patient is sensitive to insulin, the sensitivity factor may increase.

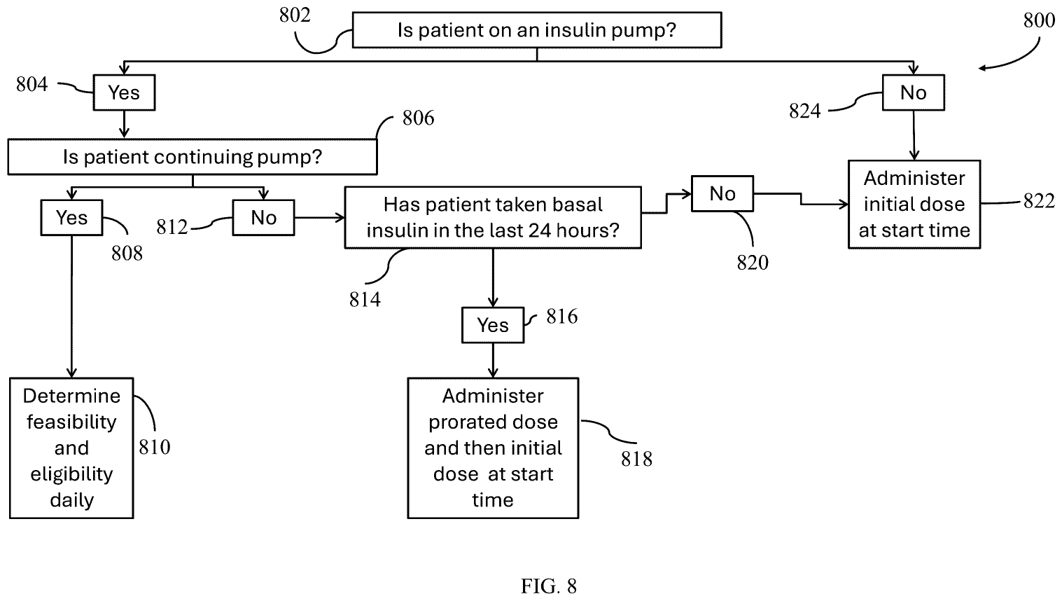

FIG. 8 shows a sequence 800 of determining an initial dose to patient. In some embodiments, system 900 may prompt user device 932 or query electronic health records (EHR) 934 to determine if a patient is on an insulin pump 802. If the patient is on an insulin pump 804, the healthcare provider may determine if the patient is to continue on the pump 806. If yes 808, system 900 and the healthcare provider may continuously determine eligibility for continuing with the treatment 810 administered by the system and the feasibility of using the insulin pump in the hospital (hospital guidelines, patient ability to manage the pump in accordance with healthcare provider guidelines, etc.). In some embodiments, the eligibility check is automatic. In some embodiments, the eligibility check is a daily eligibility check.

If the patient is not continuing on a pump 812, system 900 may prompt user device 932 or query electronic health records (EHR) 934 to determine the last time the patient received basal insulin 814, including Lantus, Tresiba, Toujeo, or Levemir, within a predetermined period of time. For example, the system may seek to determine if the patient has received a basal insulin infusion within the last 24 hours. If the patient has received a dose within the predetermined period of time 816, system 900 may administer a prorated dose and then start the initial does at a start time 818. If the patient has not received basal insulin in the last 24 hours 820, or if the patient is not on an insulin pump 824, system 900 may administer the initial dose at a start time 822. In some embodiments, the start time may be 9 PM.

In some embodiments, system 900 may prompt user device 932 or query electronic health records (EHR) 934 to determine if the patient was admitted due to hypoglycemia. If yes, and if glucose has been administered and blood sugar is at least 150, system 900 may be configured to check the blood sugar a period of time, for example, four hours later, to start the basal dose. If glucose has not been administered and blood sugar is at least 150, system 900 will continue with the care procedure described herein. Otherwise, system 900 will wait to begin the disclosed care procedure until blood sugar reaches at least 150.

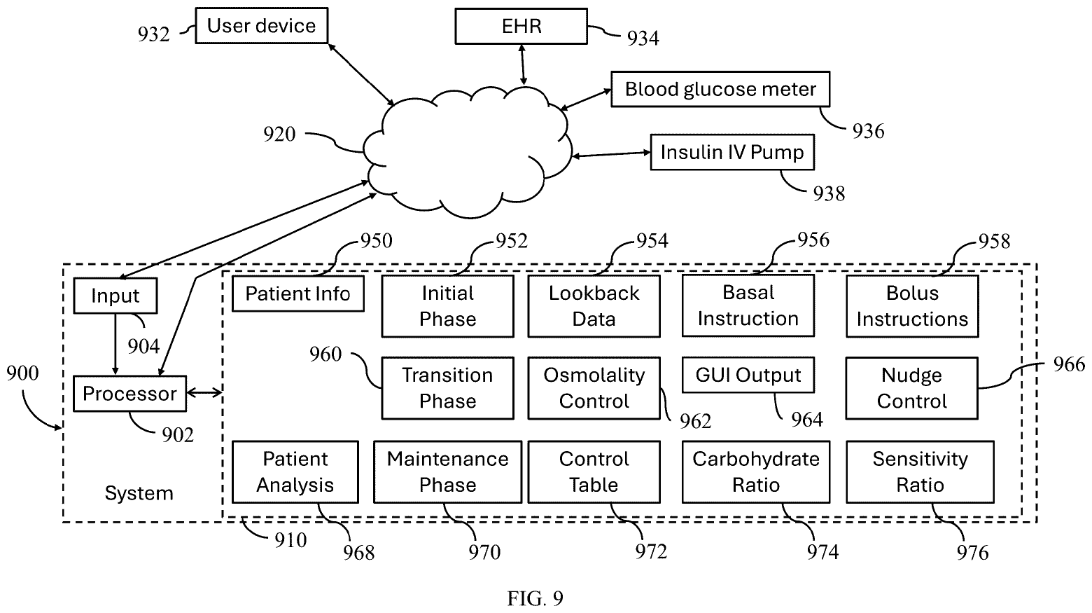

FIG. 9 shows a system 900 according to some embodiments. System 900 has processor 902. Processor 902 is configured to run modules stored in memory 906. Processor 902 is also configured to receive inputs from input module 904. Input module 904 is communicatively coupled to network 920. Input module 904 is configured to send prompts (i.e. “nudges”) to user device 932 and query EHR 934, and also receive inputs from user device 932, EHR 934, blood glucose meter 936, and insulin IV pump 938. In some embodiments, both blood glucose meter 936 and insulin IV pump 938 may be operatively coupled to the patient.

System 900 includes memory 910 that includes several modules. Each module, when executed by processor 902, causes the processor to perform a set of functions. For example, patient info module 950 may be configured to cause processor 902 to query EHR 934 to determine information about a patient. This could include biographical data, prior treatment history, comorbidities, or other information specific to the patient. Initial phase module 952 may be configured to provide instructions to processor 902 about applying an initial phase of treatment. In some embodiments, lookback data module 954 may be configured to store and provide analysis of lookback data. Basal instruction module 956 may be configured to provide basal dosing instructions to insulin IV pump 938. Bolus instruction module 958 may be configured to provide bolus instructions to a bolus injection system or insulin IV pump.

Transition phase module 960 may be configured to provide instructions to processor 952 about applying an initial phase of treatment. Osmolality control module 962 may be configured to monitor the patient's osmolality. In some embodiments, osmolality control module 962 may be configured to alert a user via user device 932 about unsafe osmolality levels of the patient. In some embodiments, the osmolality control module 962 may be configured to provide an osmolar guide to restrict changes administered glucose to ensure osmolality levels remain in a tolerable range.

GUI output module 964 may be configured to generate graphical user interfaces for user device 932. Nudge control 966 may be configured to prompt—or “nudge”—a healthcare provider to remind them of an upcoming task or a change in the patient's condition requiring their attention. Patient analysis module 968 may be configured to analyze aspects of the patient's care. For example, patient analysis module 968 may be used to compare received patient information, patient care characteristics (i.e., carbohydrate ratios or insulin sensitivity ratios), or patient care history to reference profiles to aid in predicting patients'outcomes with respect to glucose administration.

Maintenance phase module 970 may be configured to provide instructions to processor 902 about applying an initial phase of treatment. Control table module 972 may be configured to provide a control table appropriate for the patient. Carbohydrate ratio module 974 may be configured to determine a carbohydrate ratio for patient care. Sensitivity ratio module 976 may be configured to determine a sensitivity ratio for patient care.

In some embodiments, system 900 may be configured to provide outputs for a healthcare provider to administer. For example, system 900 may provide a current recommended TDD for insulin IV pump 938. Healthcare provider will enter the TDD rate in to insulin IV pump 938.

In some embodiments, system 900 for administering insulin to a patient to achieve and maintain euglycemia may prompt the healthcare provider at user device 932 or query EHR 934 to see if the patient is on oral medications, such as sulfonylureas like glyburide or glimepiride. If the patient took medication within the predetermined period of time, 24 hours, for example, system 900 may start the basal dose only after the blood sugar reading falls below 150. If the patient is not on oral medications, system 900 may proceed to the next step, and system 900 will follow the control table's initial values for managing the patient's care.

In some embodiments, system 900 may work relative to a set time. For example, 9 PM. The set time may be useful to coordinate daily checks of the patient and treatment plan. If the patent is admitted after a set time, system 900 may calculate the number of minutes until the set time, the TGM. If the TGM is greater than 180 minutes, system 900 may set a prorated basal dose of TGM/1440 multiplied by the calculated dose above (TBDD). That is, system 900 sets the prorated dose to (TBDD*TGM)/1440. If the patient receives treatment at the set time, system 900 sets the prorated basal dose to 0.

In some embodiments, system 900 may prompt the healthcare provider at user device 932 or query EHR 934 to see if the patient is on steroids. If so, system 900 may follow a steroid-stair-step for Humalog dosing. In some embodiments, system 900 may prompt the healthcare provider at user device 932 or query EHR 934 to see if the patient is on home medications includes medications that are insulin sensitizers (pioglitazone, GLP-1 Agonists—ozempic, trulicity, victoza, saxenda, bydureon, soliqua). If so, then column 4 of the control table for prorated/initial doses may be disregarded by the system.

In some embodiments, system 900 may prompt the provider to suggest that the patient not be given metformin in the hospital while the patient's glucose levels are being measured and managed by the system. In some embodiments, if the patient is on DPP-4, system 900 may suggest replacing the DPP-4 with Humalog while in the hospital, with the DPP-4 restarting on discharge. If the patient is on SGL-T2 inhibitors, follow HCO3/AG/CO2 levels. In some embodiments, if the patient is a type 1 diabetic, system 900 will not discontinue the basal insulin.

In some embodiments, the system may be configured to take the patient's blood sugar at regular intervals, for example, every 15 minutes. In some embodiments, the system may be configured to sample the patient's blood sugar at defined times, such as, for example, after fasting (6 AM), before lunch, before dinner, bedtime (9 PM), and early morning (3 AM). In some embodiments, the system may include an alarm. The alarm may be triggered if one or more measurements are outside a defined range. For example, renal failure is indicated when serum creatinine is greater than or equal to 1.8. To maintain glucose within target range while minimizing hypoglycemia, the algorithm continuously updates dosing parameters based on observed insulin sensitivity and carbohydrate ratio variability. This adaptive approach enables tight yet safe glucose control throughout hospitalization. Additionally, the insulin regimen developed during the inpatient stay provides a framework for improved glycemic management after discharge.

In some embodiments, the processor is applying dosing algorithms. In those circumstances, the processor 902 may be configured to have the IV pump 938 apply both a basal and a bolus component of the dosage. In some embodiments, the bolus component is administered by a healthcare provider or another system at the direction of the processor. In some embodiments, the processor applies different rules to the basal and bolus components. The bolus component is fast acting to address rapid shifts in a patient's blood sugar, for example, after eating. In contrast, the basal component is the steady of insulin to address the patient's needs.

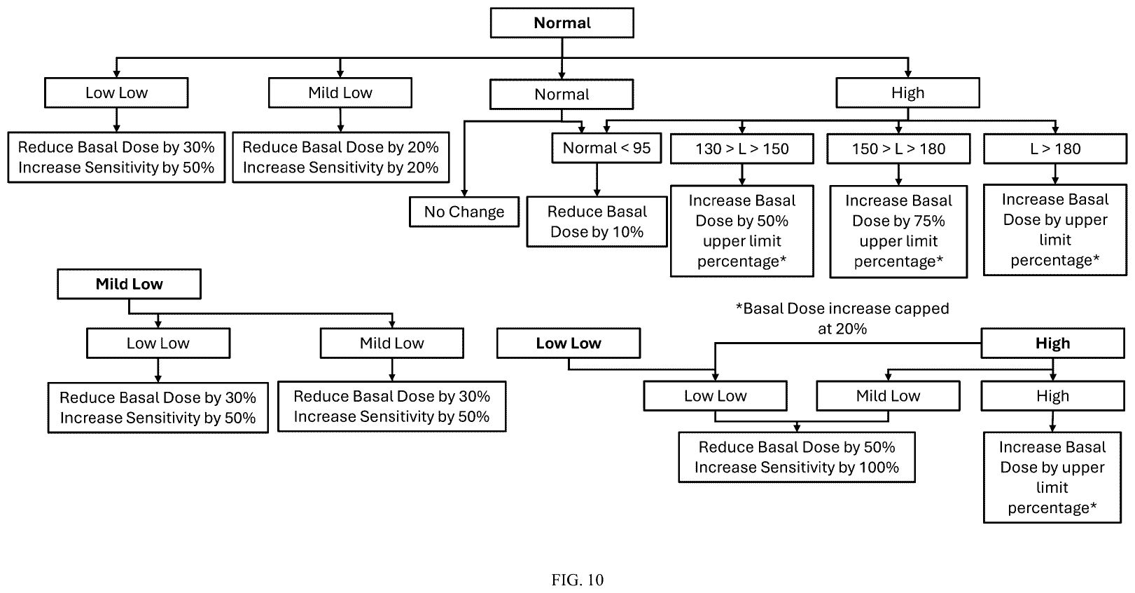

In some embodiments, system 900 may be configured to administer a second basal dose. The method applied by system 900 to control patient glycemia may include accounting for all four columns of the control table and focusing on the 3 AM and 6 AM blood sugar levels. The blood sugar ranges may be defined as follows: normal is between 80 and 130, high is above 130, mild low is between 65 and less than 80, and low low is below 65. A decision tree is shown in FIG. 10, and described below.

The blood sugar levels of the patient are taken at 3 AM and 6 AM. If both are normal or if one is high and the other is normal, and if the normal level is less than 95, the basal dose should be reduced by 10%. Otherwise, no change is needed. If both levels are high, the basal dose should be increased by the upper limit percentage from the control table. If one level is normal and the other is high, the adjustment depends on the high blood sugar level: if it is between 130 and 150, the basal dose should be changed by 0.5 times the upper limit percentage; if it is between 150 and 180, the change should be 0.75 times the upper limit percentage; and if it is above 180, the change should be 1 times the upper limit percentage. However, if the change in basal dose exceeds 20%, it should be capped at 20%.

When both levels are mild low, the basal dose should be reduced by 30% and sensitivity increased by 50% in the control table. If both levels are low low, or if one is low low and the other is high, or if one is high and the other is low low, the basal dose should be reduced by 50% and sensitivity increased by 100% in the control table. If one level is normal and the other is mild low, or vice versa, the basal dose should be reduced by 20% and sensitivity increased by 20% in the control table. If one level is low low and the other is normal or mild low, or vice versa, the basal dose should be reduced by 30% and sensitivity increased by 50% in the control table.

For subsequent basal doses, the average between the calculated dose from the control table and the observed dose should be used. If the basal dose exceeds 60% of the total daily dose, the overflow should be distributed evenly among Humalog doses. The Humalog dose includes correction (given at the time of blood sugar testing), meal, and overflow (given at the time of the meal). The observed change in basal dose should be compared with the calculated change based on the control table. If the observed change is less than or equal to the calculated change, the observed change should be followed. Otherwise, the basal dose should be set to the average of the observed and calculated changes. If the new basal dose exceeds 0.5 units per kilogram, the increase should be spread between basal and bolus doses.

In some circumstances, a patient's care may include a basal and bolus insulin component. The disclosed insulin dosing algorithm is designed to achieve glycemic control through a combination of basal and bolus components. The bolus component is fast-acting to address rapid shifts in a patient's blood sugar, for example, after eating. In contrast, the basal component is the steady state of insulin administered to address the patient's needs.

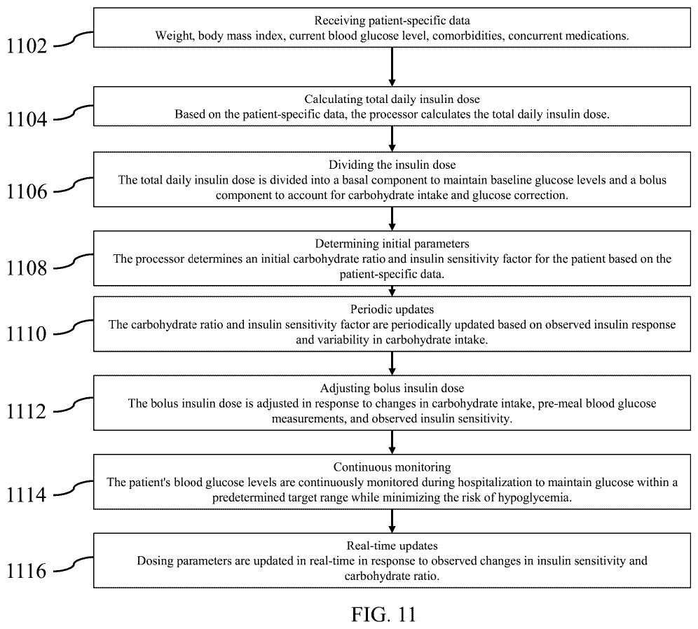

A method of administering a basal bolus protocol to a patient is described below with reference to FIG. 11. In some embodiments, method 1100 includes receiving patient-specific data 1102. In some embodiments, this includes weight, body mass index, current blood glucose level, comorbidities, concurrent medications. Method 1100 may also include calculating total daily insulin dose 1104 based on the patient-specific data. In some embodiments, method 1100 includes dividing the insulin dose 1106. In some embodiments, the total daily insulin dose is divided into a basal component to maintain baseline glucose levels and a bolus component to account for carbohydrate intake and glucose correction. Method 1100 may include determining initial parameters 1108. This includes determining an initial carbohydrate ratio and insulin sensitivity factor or ratio for the patient based on the patient-specific data. In some embodiments, method 1100 includes periodic update 1110. This periodically updates the carbohydrate ratio and insulin sensitivity factor updated based on observed insulin response and variability in carbohydrate intake.

Method 1100 may also include adjusting bolus insulin dose 1112. The bolus insulin dose is adjusted in response to changes in carbohydrate intake, pre-meal blood glucose measurements, and observed insulin sensitivity. In some embodiments, method 1100 includes continuous monitoring 1114. This may include the continuous monitoring of the patient's blood glucose levels during hospitalization. This is important to maintain glucose within a predetermined target range while minimizing the risk of hypoglycemia. In some embodiments, method 1100 includes real-time updates 1116. Real-time update 1116 may include providing updates to dosing parameters in real-time in response to observed changes in insulin sensitivity and carbohydrate ratio.

In some embodiments, a Humalog dose is the total dose, which is the sum of the correction dose and the meal (carb) dose. The correction dose may be calculated as the difference between the point-of-care blood glucose (POC BG) and the target blood glucose, divided by the sensitivity factor. In some embodiments, the method includes adjusting the target blood glucose to avoid insulin stacking: if Humalog was given within less than 2 hours, the target is 200; if given within 2-3 hours, the target is 150; if given within 3-4 hours, the target is 125; and if given more than 4 hours ago, the default target is 100. If the POC BG is above 250 mg/dL, the correction dose should be given immediately. If the POC BG is below 250 mg/dL and the patient is eating soon, the correction and meal doses should be given together. If the patient is not eating yet, only the correction dose should be given. When combining correction and meal doses, the POC BG must be within one hour of eating.

The meal dose is determined by the carbs eaten divided by the carb ratio. Carbs eaten can be calculated as the carbs served minus the carbs left over, or as the percentage eaten multiplied by the fixed carbs served for fixed carb meals. For high-protein, low-carb meals, the system may be configured to use a post-meal dose of 0.4 times the protein grams eaten, or a 2-hour post-meal correction dose based on the sensitivity factor. The preferred timing for dosing is pre-meal if the full meal is expected to be eaten, or post-meal if food intake is uncertain.

To adjust sensitivity and basal dose using prior day data, the average correction dose used the previous day should be calculated. The adjustment dose is split, with 50% used to adjust the basal dose and 50% to recalculate the sensitivity factor and carbohydrate ratio. For snacks and off-schedule intake, additional insulin should only be given if the insulin dose via the carb ratio exceeds 1 unit, and insulin stacking should be avoided by following the stacking protocol.

For steroid-induced hyperglycemia, if the patient is on once-daily steroids like prednisone, the carb ratio adjustment is as follows: for breakfast, use the standard correction and carb ratio; for lunch, use 75% of the baseline carb ratio; and for dinner, use 50% of the baseline carb ratio. Baseline ratios should be promptly reverted upon steroid discontinuation. For transitioning from IV to subcutaneous insulin, the basal dose should be administered 2 hours before stopping IV insulin, and for pump transition, a subcutaneous bolus should be given 30 minutes before stopping IV insulin.

In some embodiments, the disclosed systems utilize an adaptive algorithm to dynamically adjust insulin infusion rates based on real-time and historical patient data. This algorithm is designed to achieve and maintain euglycemia while minimizing the risk of hypoglycemia and hyperglycemia, particularly in critical care settings.

The adaptive algorithm operates across three primary phases—initial, transition, and maintenance—and incorporates a proportional-integral-derivative (PID) control model. It continuously evaluates patient-specific inputs and glucose trends to determine optimal drip rates.

Inputs to the algorithm may include: patient-specific data: weight, body mass index (BMI), comorbidities, concurrent medications, and osmolality; real-time glucose readings: obtained via point-of-care meters or continuous glucose monitors (CGMs); historical glucose trends: captured over a defined lookback period (e.g., four consecutive readings); prior insulin responses: including previous drip rates and their effects on glucose levels.

Algorithm logic and phase transitions may include, during the initial phase: begins with a calculated drip rate based on current glucose and patient weight; predicts glucose level at time Tn (e.g., 1 hour later) using the current drip rate; compares predicted vs. observed glucose levels; adjusts drip rate based on deviation and trend direction; continues until glucose falls below a predetermined threshold (e.g., 200 mg/dL). During the transition phase, the algorithm logic may include: using moderated drip rate adjustments to slow the rate of glucose decline; apply similar predictive logic as the initial phase but with reduced aggressiveness; and ends when glucose stabilizes within a target range (e.g., 100-140 mg/dL). During the maintenance phase, the algorithm logic may: evaluate glucose readings over a lookback period (e.g., four readings); detect directional trends (e.g., all increasing or decreasing); calculate the cumulative magnitude of change; adjust the drip rate proportionally: if glucose is rising and exceeds thresholds, increases drip rate and if glucose is falling and below thresholds, decreases drip rate. The algorithm logic may incorporate safety overrides for hypoglycemia (e.g., drip rate set to zero if glucose <80 mg/dL).

In some embodiments, the systems disclosed herein are configured to refine its predictions and actions over time by updating insulin sensitivity factors and carbohydrate ratios based on observed responses. In some embodiments, the system may be configured to weight recent glucose readings more heavily in trend analysis, recognize patterns linked to medications, meals, or stressors, and adjust dosing logic accordingly to improve accuracy and responsiveness.

The disclosed embodiments improve insulin infusion by coupling adaptive control logic with specific infusion hardware and clinical data sources. A processor receives a real-time blood glucose readings from a blood glucose meter. The processor may also query, receive, and adapt information in an electronic health records database. A dosing instruction, determined by the processor using algorithms disclosed herein, is transmitted to an insulin IV pump. The system implements safety overrides at the device-control level (e.g., setting the drip rate to 0 units/hour in a hypoglycemic mode when glucose <80 mg/dL) and incorporates osmolality-based constraints that suspend or limit insulin delivery under hyperosmolar conditions until safe thresholds are re-established.

The adaptive control strategy regulates how insulin is delivered over time. During an initial phase, the controller predicts glucose at time Tn based on the active drip rate, compares the observed and predicted values, and updates the rate to quickly drive glucose toward a predetermined threshold above euglycemia; the transition phase then moderates the rate of decline to provide a “safe landing” into a euglycemic target range. In the maintenance phase, the controller evaluates a defined lookback period (e.g., four consecutive readings across about 2-4 hours), verifies that successive readings move in the same direction, and applies direction-specific cumulative-magnitude thresholds (e.g., ≥10% decrease to reduce the drip rate; ≥15% increase to augment the drip rate) before any change is permitted.

The disclosed systems improve clinical workflows and patient safety by automating burdensome tasks, personalizing therapy, and providing safety guardrails for changes in insulin administration. The controller continually updates dosing parameters (e.g., sensitivity factors and carbohydrate ratios) and generates GUI outputs that display patient-specific data and drip-rate history for clinician verification. The system coordinates transitions by calculating a subcutaneous basal dose based on observed IV requirements and administering it with a defined overlap before discontinuing IV infusion, and it can account for medications (e.g., steroids or insulin sensitizers) and set-time scheduling (e.g., 9 PM) via protocolized logic and clinician “nudges.”

In some embodiments, the disclosed system employs adaptive infusion logic that integrates trend-based control with proportional-integral-derivative (PID) principles to dynamically tailor insulin delivery to patient-specific conditions. Unlike static feedback loops that react only to the most recent glucose reading, the adaptive algorithm evaluates directional trends over a defined lookback period, calculates cumulative magnitude of change, and applies predictive modeling to anticipate future glucose trajectories. This approach enables the processor to adjust drip rates proactively rather than reactively, reducing oscillations and minimizing the risk of overshoot into hypoglycemia or rebound hyperglycemia. The adaptive logic operates in real time, continuously recalibrating based on observed deviations from predicted glucose levels, rate of change, and cumulative error, thereby improving dosing accuracy and stability across all phases of infusion.

The adaptive control is implemented within a hardware-integrated architecture comprising a blood glucose sensor (e.g., point-of-care meter or continuous glucose monitor), an insulin IV pump, a processor executing the dosing algorithm, and an electronic health records (EHR) interface for contextual data. This combination allows the system to incorporate patient-specific variables such as weight, comorbidities, concurrent medications, and osmolality constraints into its decision-making process. Over time, the algorithm refines its internal parameters—such as insulin sensitivity factors and carbohydrate ratios—through machine learning techniques that recognize patterns linked to medications, stress states, or meal timing. By coupling predictive analytics and AI-driven trend detection with direct control of infusion hardware, the disclosed system achieves a specific technological improvement: faster attainment of euglycemia, sustained glucose stability, and reduced incidence of hypo-and hyperglycemic events in critical care settings.

The present disclosure uses examples to improve the understanding of the present disclosure. It should be noted that steps may be added or omitted without deviating from the scope of the present disclosure. The thresholds, multipliers, and other values may be changed without deviating from the scope of the present disclosure.

Many additional implementations are possible. Further implementations are within the CLAIMS.

It will be understood that implementations of the systems and methods for insulin infusion include but are not limited to the specific components disclosed herein, as virtually any components consistent with the intended operation of various systems and methods for insulin infusion may be utilized.

The concepts disclosed herein are not limited to the specific systems and methods for insulin infusion shown herein. In places where the description above refers to particularities in insulin infusion implementations, it should be readily apparent that a number of modifications may be made without departing from the spirit thereof and that these implementations may be applied to other implementations disclosed or undisclosed. The presently disclosed systems and methods for insulin infusion are, therefore, to be considered in all respects as illustrative and not restrictive.

What is claimed is:

1. A method of administering insulin to a patient to achieve and maintain euglycemia, the method comprising:
administering insulin to the patient at a drip rate that is based on a current blood glucose level of the patient and a weight of the patient;
during an initial phase, adjusting the drip rate periodically until a blood glucose level of the patient decreases to a predetermined blood glucose level above euglycemia, wherein adjusting the drip rate during the initial phase comprises repeatedly:
determining a target blood glucose level for time Tₙ based on the current drip rate;
observing the blood glucose level at time Tₙ;
comparing the observed blood glucose level with the predetermined blood glucose level;
comparing the observed blood glucose level with the target blood glucose level; and
calculating an updated drip rate based on the comparison of the observed blood glucose level with the target blood glucose level;
during a transition phase, adjusting the drip rate such that the blood glucose level of the patient decreases slower during the transition phase than the initial phase; and
after the blood glucose level reaches euglycemia during the transition phase, entering a maintenance phase;
during the maintenance phase, adjusting the drip rate periodically based on a current drip rate and a percentage change in blood glucose level during a period of time; and
during the maintenance phase:
evaluating all blood glucose levels of the patient measured that occurred during a lookback period;
determining whether each successive blood glucose level measurement moved in a same direction;
determining a cumulative magnitude of change in blood glucose level during the lookback period;
determining a current blood glucose level;
increasing the drip rate if each successive blood glucose level measurement during the lookback period increased, the current blood glucose level is above a predetermined level, and the cumulative magnitude of change is above a predetermined magnitude; and
decreasing the drip rate during the lookback period if each successive blood glucose level measurement during the lookback period decreased, the current blood glucose level is below a predetermined level, and the cumulative magnitude of change is above a predetermined magnitude.

2. The method of claim 1, wherein the lookback period comprises a four-period period.

3. The method of claim 1, wherein the predetermined blood glucose level is 200 mg/dL.

4. The method of claim 1, wherein an amount of time in the initial phase is predetermined.

5. The method of claim 1, wherein the initial phase lasts four hours.

6. The method of claim 1, wherein an amount that the blood glucose level of the patient decreases during the initial phase is limited based on an osmolality of the patient.

7. The method of claim 1, wherein a basal insulin dosage for subcutaneous delivery is calculated at an end of the maintenance phase.

8. The method of claim 1, further comprising administering a bolus insulin dosage in response to a change in carbohydrate intake.

9. The method of claim 1, further comprising determining a patent glucose correction, and administering a bolus insulin dosage in response to the determined patient glucose correction.

10. The method of claim 9, wherein the bolus insulin dosage is administered with the basal component.

11. A system for administering insulin to a patient to achieve and maintain euglycemia, the system comprising:
an insulin IV pump;
a server communicatively coupled to:
a user device;
a blood glucose meter;
an electronic health records database; and
the insulin IV pump; and
a processor communicatively coupled to the server, the processor configured to:
receive user inputs from the user device;
receive a blood glucose reading from the blood glucose meter;
receive electronic health records from the electronic health records database;
send a drip rate instruction to the insulin IV pump;
wherein the drip rate instruction to the insulin IV pump has an initial drip rate instruction, an initial phase instruction, a transition phase instruction, and a maintenance phase instruction,
wherein the initial drip rate instruction is based on a current blood glucose reading received from the blood glucose meter and a patient weight received from the user device,
wherein the initial phase instruction comprises periodically adjusting the drip rate by:
determining a target blood glucose level for time Tn based on a current drip rate;
receiving an observed blood glucose level at time Tn;
comparing the observed blood glucose level with a predetermined blood glucose level above euglycemia;
comparing the observed blood glucose level with the target blood glucose level; and
calculating an updated drip rate based on the comparison of the observed blood glucose level with the target blood glucose level;
wherein the transition phase instruction comprises adjusting the drip rate such that the blood glucose level of the patient decreases at a slower rate during the transition phase than the initial phase,
wherein the maintenance phase instruction comprises adjusting the drip rate periodically based on the current drip rate and a percentage change in blood glucose level during a period of time, and
wherein, during the maintenance phase, the processor is configured to:
evaluating all blood glucose levels of the patient measured that occurred during a lookback period;
determining whether each successive blood glucose level measurement moved in a same direction;
determining a cumulative magnitude of change in blood glucose level during the lookback period;
determining a current blood glucose level;
increasing the drip rate if each successive blood glucose level measurement during the lookback period increased, the current blood glucose level is above a predetermined level, and the cumulative magnitude of change is above a predetermined magnitude; and
decreasing the drip rate during the lookback period if each successive blood glucose level measurement during the lookback period decreased, the current blood glucose level is below a predetermined level, and the cumulative magnitude of change is above a predetermined magnitude.

12. The system of claim 11, wherein the processor is further configured to query the electronic health records database to determine if the patient has received a basal insulin formulation within the previous 24 hours.

13. The system of claim 12, wherein the processor is further configured to:
a) if the previous dose was administered within the last 24 hours, instruct the IV pump administer an initial basal dose at set time; and
b) if the previous dose was not administered within the last 24 hours, instructing the iv p to administer a prorated basal dose and subsequently administering the initial basal dose at the set time.

14. The system of claim 11, wherein the processor is configured to receive a patient osmolality.

15. The system of claim 14, wherein the processor is configured to adjust the dosage based on the received patient osmolality.

16. The system of claim 11, wherein the lookback period comprises a four-period period.

17. The system of claim 11, wherein the processor is further configured to generate a graphical user interface having:
a first section displaying patient-specific data; and
a second section displaying a chart of the drip rate over time.

18. A method of administering insulin to a patient to achieve and maintain euglycemia, the method comprising:
receiving patient-specific data;
determining an initial carbohydrate ratio and an initial insulin sensitivity factor based on the patient-specific data;
calculating a total daily insulin dose based on the patient-specific data;
dividing the total daily insulin dose into a bolus component and a basal component, the basal component comprising an initial drip rate;
periodically updating the carbohydrate ratio and the insulin sensitivity factor based on an observed insulin response, a carbohydrate intake, and a lookback period; and
continuously adjusting the bolus component in response to changes in carbohydrate intake and the observed insulin sensitivity.

19. The system of claim 18, wherein the lookback period comprises a four-period period.

20. The system of claim 18, the patient-specific data comprises weight, body mass index, current blood glucose level, comorbidities, and information regarding concurrent medications.
